(19) United States
(12) Patent Application Publication
(10) Pub. No.: US 2022/0163512 A1
(43) Pub. Date: May 26, 2022

(54) BETA-THALASSEMIA POTENCY ASSAY

(71) Applicant: bluebird bio, Inc., Cambridge, MA (US)

(72) Inventors: ILYA SHESTOPALOV, BILLERICA, MA (US); MELISSA BONNER, NATICK, MA (US)

(73) Assignee: bluebird bio, Inc., Cambridge, MA (US)

(21) Appl. No.: 17/598,637

(22) PCT Filed: Mar. 27, 2020
(86) PCT No.: PCT/US2020/025503

§ 371 Date: Sep. 27, 2021

Related U.S. Application Data
(60) Provisional application No. 62/824,996, filed on Mar. 27, 2019.

(51) Int. Cl. G01N 33/50; C12N 15/86; C12N 5/078; G01N 33/80; C07K 14/795

(52) U.S. Cl. CPC G01N 33/5023; C12N 15/86; C12N 5/0641; C12N 2506/03; C07K 14/795; C12N 2740/15043; G01N 33/80

(57) ABSTRACT

Disclosed herein are potency assays for a gene therapy treatment for β-thalassemia. Also disclosed herein are methods for measuring relative potency of a drug product.

### RELATED APPLICATIONS

This application claims the benefit of U.S. Provisional Application No. 62/824,996, filed Mar. 27, 2019. The entire teachings of the above application are incorporated herein by reference.

### BACKGROUND OF THE INVENTION

In β-thalassemia major, genetic mutations diminish or completely abrogate β-globin expression, resulting in accumulation of monomeric α-globin during erythroblast differentiation. This globin chain imbalance results in cellular stress and apoptosis. Defective erythropoiesis becomes evident by attrition of erythroblasts starting at the polychromatophilic stage, and the few differentiated erythrocytes either get trapped in the bone marrow or exhibit short lifespan in circulation. β-thalassemia patients therefore rely on transfusions for survival.

Consistent with the disease, CD34⁺ cells deficient for β-globin have inhibited erythroid differentiation potential in culture, as measured by percent abundance of enucleated cells and acquisition of mature erythrocyte phenotype (CD235⁺/CD71⁻). Lentiviral integration of a transgene expressing β-globin from an erythroid-specific promoter into CD34⁺ cells balances α-globin expression, resulting in production of healthy erythroblasts and transfusion independence following autologous transplantation into β-thalassemia patients. As therapies for treating β-thalassemia progress, there is a need for methods by which drug product potency may be measured and quantified.

### SUMMARY OF THE INVENTION

Disclosed herein are potency assays for a gene therapy treatment for β-thalassemia. The potency assays comprise: transducing a sample of hematopoietic stem or progenitor cells from a subject having β-thalassemia with a lentiviral vector comprising a polynucleotide encoding a globin; erythroid differentiating the transduced hematopoietic stem or progenitor cells; erythroid differentiating a sample of untransduced hematopoietic stem or progenitor cells from the subject having β-thalassemia; measuring fold change in Hemoglobin A expression in the transduced and the untransduced erythroid cell samples; and measuring fold change in enucleated reticulocytes in the transduced and the untransduced erythroid cell samples, wherein the potency of the gene therapy is assessed as the fold change in HbA expression and/or fold change in percent enucleated reticulocytes, in the transduced compared to the untransduced erythroid cell samples.

Also disclosed herein are potency assays for a gene therapy treatment for β-thalassemia. The potency assays comprise transducing a sample of hematopoietic stem or progenitor cells from a subject having β-thalassemia with a lentiviral vector comprising a polynucleotide encoding a globin; erythroid differentiating the transduced hematopoietic stem or progenitor cells; erythroid differentiating a sample of untransduced hematopoietic stem or progenitor cells from the subject having β-thalassemia; and measuring fold change in Hemoglobin A expression in the transduced and the untransduced erythroid cell samples, wherein the potency of the gene therapy is assessed as the fold change in HbA expression in the transduced compared to the untransduced erythroid cell samples.

Also disclosed herein are potency assays for a gene therapy treatment for β-thalassemia. The potency assays comprise transducing a sample of hematopoietic stem or progenitor cells from a subject having β-thalassemia with a lentiviral vector comprising a polynucleotide encoding a globin; erythroid differentiating the transduced hematopoietic stem or progenitor cells; erythroid differentiating a sample of untransduced hematopoietic stem or progenitor cells from the subject having β-thalassemia; and measuring fold change in enucleated reticulocytes in the transduced and the untransduced erythroid cell samples, wherein the potency of the gene therapy is assessed as the fold change in percent enucleated reticulocytes in the transduced compared to the untransduced erythroid cell samples.

In some embodiments, the potency assay further comprises obtaining the hematopoietic stem or progenitor cells from the subject that has β-thalassemia. In some embodiments, the hematopoietic stem or progenitor cells comprise CD34⁺ cells, CD133⁺ cells, or CD34⁺CD38^{Lo}CD90⁺CD45RA⁻ cells. In some embodiments, the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺. In some embodiments, the globin is a human β-globin, an anti-sickling globin, a human β^{A-T87Q}-globin, a human β^{A-G16D/E22A/T87Q}-globin, or a human β^{A-T87Q/K95E/K120E}-globin protein. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a GLOBE vector, a G-GLOBE vector, a βAS3-FB vector, or a derivative thereof.

In some embodiments, the erythroid differentiation method comprises a two-stage culture. In some embodiments, the erythroid differentiation method occurs for a period of 14-18 days or 14-17 days.

In some embodiments, the fold change in Hemoglobin A expression is measured using ion-exchange HPLC. In some embodiments, the fold change in enucleated reticulocytes is measured using FACS.

Disclosed herein are methods for measuring relative potency of a drug product. The methods comprise transducing a sample of hematopoietic stem or progenitor cells from the subject having β-thalassemia and erythroid differentiating the transduced cells; erythroid differentiating a sample of untransduced hematopoietic stem or progenitor cells from the subject having β-thalassemia; quantifying fold change in Hemoglobin A (HbA) expression in the transduced erythroid cells compared to the HbA expression in the untransduced erythroid cells; and quantifying fold change in the number of enucleated reticulocytes in the transduced erythroid cells compared to the number of enucleated reticulocytes in the untransduced cells, wherein the transduced erythroid cells contain a lentiviral vector comprising a polynucleotide encoding a globin.

Also disclosed herein are methods for measuring relative potency of a drug product. The methods comprise transducing a sample of hematopoietic stem or progenitor cells from the subject having β-thalassemia and erythroid differentiating the transduced cells; erythroid differentiating a sample of untransduced hematopoietic stem or progenitor cells from the subject having β-thalassemia; and quantifying fold change in Hemoglobin A (HbA) expression in the transduced erythroid cells compared to the HbA expression in the untransduced erythroid cells, wherein the transduced erythroid cells contain a lentiviral vector comprising a polynucleotide encoding a globin.

Also disclosed herein are methods for measuring relative potency of a drug product. The methods comprise transducing a sample of hematopoietic stem or progenitor cells from the subject having β-thalassemia and erythroid differentiating the transduced cells; erythroid differentiating a sample of untransduced hematopoietic stem or progenitor cells from the subject having β-thalassemia; and quantifying fold change in the number of enucleated reticulocytes in the transduced erythroid cells compared to the number of enucleated reticulocytes in the untransduced cells, wherein the transduced erythroid cells contain a lentiviral vector comprising a polynucleotide encoding a globin.

In some embodiments, the methods further comprise obtaining the hematopoietic stem or progenitor cells from the patient having β-thalassemia. In some embodiments, the hematopoietic stem or progenitor cells comprise CD34⁺ cells, CD133⁺ cells, or CD34⁺CD38^{LO}CD90⁺CD45RA⁻ cells. In some embodiments, the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{E}/β⁺, β⁰/β⁺, and β⁺/β⁺. In some embodiments, the globin is a human β-globin, an anti-sickling globin, a human β^{A-T87Q}-globin, a human β^{A-G16D/E22A/T87Q}-globin, or a human β^{A-T87Q/K95E/K120E}-globin protein. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a GLOBE vector, a G-GLOBE vector, a βAS3-FB vector, or a derivative thereof.

In some embodiments, the fold change in Hemoglobin A expression is measured using ion-exchange HPLC. In some embodiments, the fold change in enucleated reticulocytes is measured using FACS.

Disclosed herein are potency assays for a gene therapy treatment for β-thalassemia. The potency assays comprise transducing a first sample of hematopoietic stem or progenitor cells from a subject having β-thalassemia with a lentiviral vector comprising a polynucleotide encoding a globin; performing erythroid differentiation of the first sample of hematopoietic stem or progenitor cells; performing erythroid differentiation of a second sample of untransduced hematopoietic stem or progenitor cells from the subject having β-thalassemia; measuring fold change in Hemoglobin A expression in the transduced and the untransduced erythroid cell samples; and measuring fold change in enucleated reticulocytes in the transduced and the untransduced erythroid cell samples, wherein the potency of the gene therapy is assessed as the fold change in HbA expression and/or fold change in percent enucleated reticulocytes, in the first sample compared to the second sample.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1B provide schematics of erythropoiesis and hemoglobin expression. FIG. 1A provides a schematic of erythropoiesis showing generation of RBCs from CD34⁺ HSPCs in the BM. Erythropoiesis takes about 3.5 weeks in vivo.

Hemoglobin gene expression begins after 10 days of differentiation. Enucleation is the condensation and excretion of DNA to form reticulocytes. FIG. 1B provides a schematic of an in vitro erythropoiesis model to induce HbA^{T87Q} expression. Drug product CD34⁺ cells differentiate into erythroblasts, express HbA^{T87Q}, and form reticulocytes and RBCs.

FIG. 2 demonstrates that HbA^{T87Q} corrects arrest at enucleation step in β-thalassemia. Potency of a drug product can be measured as a relative increase in % enucleated cells.

FIG. 3 shows HbA^{T87Q} expression rescues enucleation in β-thalassemia.

FIG. 4 demonstrates potency correlates with transgene expression levels. HbA^{T87Q} protein expression increases with vector copy number (VCN) in an optimized assay and in a sub-optimal assay. However, only the optimized assay version leads to VCN- and protein-dependent increase in enucleated cells. The optimized assay demonstrates the ability to detect sub-functional drug products

FIG. 5 shows protein expression from LVV transgene reliably corrects the enucleation defect. Across all 25 subjects, potency was above the 10% threshold at VCN >1 c/dg. Below 1 c/dg, 3 of 8 samples lacked enucleation potency.

FIGS. 6A-6E show resolution of hemoglobin tetramers by IE-HPLC. Chromatograms show absorbance at 418 nm. FIG. 6A provides reference standard AFSC (mix of HbA, HbF, HbS, HbC), with 5 labeled peaks, used to make peak assignment for all samples. HbA2 elutes as a shoulder immediately following HbA. FIG. 6B provides reference standard AA2 (mix of HbA, HbA2) with 1 labeled peaks. FIG. 6C shows healthy CD34⁺ cells prior to culture with no peaks. FIG. 6D shows cells obtained from a culturing method at day 14 from healthy CD34⁺ cells. FIG. 6E shows cells obtained from a culturing method at day 14 from β-thalassemia CD34⁺ cells.

FIGS. 7A-7C demonstrates linearity of IE-HPLC method. FIG. 7A shows reference standard AA2 (FIG. 6B) with known hemoglobin concentration was serially diluted 2-fold. Triplicate injections were performed at each concentration. HbA peak area vs hemoglobin amount and linear fit is shown. R²=0.9990. % CV: 7.43 pmol: 1.3; 3.72 pmol: 2.0; 1.85 pmol: 7.8; 0.92 pmol: 20.7. 0.46 pmol was below LOQ. FIG. 7B provides a Table demonstrating precision of IE-HPLC analysis of abundant hemoglobin component. CD34⁺ cells were cultured for 14 days and pellets of 1×10⁶ cells were frozen at −80° C. On Day 1, 3 pellets were thawed and lysed by Analyst 1 and 3 pellets were thawed and lysed by Analyst 2. Replicate cell lysis was repeated on Day 2 and Day 3. All lysates were frozen at −80° C. until IE-HPLC analysis. Chromatograms were obtained on the same day using 30 uL of each lysis replicate and integrated across the hemoglobin peak region (5-15 min). Peak abundance is reported as % HbA area of total integrated region (see FIG. 6D). FIG. 7C provides a Table demonstrating precision of IE-HPLC analysis of rare hemoglobin component. Chromatograms used to generate FIG. 7C were integrated for % HbF area (see FIG. 6D).

FIGS. 8A-8E demonstrate results of FACS-based enucleation assay. Cells were stained with nuclear dye DRAQ5. Gates are drawn based on three nucleated erythroblast populations (small, medium, large) and enucleated cells. FIG. 8A shows RBCs are 97.7% enucleated, with a small population of reticulocytes. FIG. 8B shows healthy undifferentiated CD34⁺ cells are 99% nucleated. Following differentiation for 7 days (FIG. 8C), a large erythroblast population appears. At 14 days of differentiation, enucleated cells along with small and medium erythroblasts are quantified. FIG. 8E shows cytospin of sample (FIG. 8D) confirms ˜30% enucleation. Scale bar: 50 μm.

FIG. 9 provides a Table demonstrating precision of FACS-based enucleation analysis. CD34⁺ cells were cultured for 14 days and aliquots of 5×10⁵ cells were made in PBS containing 2% FBS. At time 1, 3 cell replicates were stained with DRAQ5 by Analyst 1, and 3 cell replicates were stained by Analyst 2. The procedure was repeated at time 2 and time 3. Samples were analyzed on the BD-Accuri and of enucleated cells were quantified using FlowJo software.

FIG. 10 shows growth kinetics of healthy and β-thalassemia CD34⁺ cells using an erythroid culture method. Viable cell counts were normalized to 1×10⁶ starting cells. Day 0 is erythroid culture initiation day. In the case of transductions with an LVV encoding GFP, prestim was performed prior to day 0. Transductions were performed at MOI 25.

FIG. 11 shows effect of enucleation upon culture duration and flow-cytometer used for analysis. Two healthy lots of CD34⁺ cells and one β-thalassemia lot of CD34⁺ cells were cultured. Readouts were performed at the indicated days. The enucleated fraction was measured from the same samples using BD-Accuri and BD-Canto flow cytometers.

FIGS. 12A-12D demonstrate transduction with LentiGlobin BB305 LVV rescues HbA expression in β-thalassemia CD34⁺ cells. β-thalassemia CD34⁺ cells were either prestimulated for 48 hrs (FIG. 12A) or prestimulated for 48 hrs and transduced with LentiGlobin BB305 LVV at MOI 25 (FIG. 12B), followed by erythroid differentiation. A VCN of 0.62 was obtained from the cell culture at day 14. Cell pellets were analyzed by IE-HPLC. Peak assignment is based on AFSC hemoglobin control (FIG. 6A). Peak abundance is reported as % area of all hemoglobin peaks. FIG. 12C shows hemoglobin content of β-thalassemia CD34⁺ cells that were either freshly thawed, prestimulated for 48 hrs (as in FIG. 12A), mock transduced, or transduced with LentiGlobin BB305 LVV at MOI 25 (as in FIG. 12B), followed by erythroid differentiation for 14 days. FIG. 12D shows hemoglobin content at day 18 of erythroid differentiation. Error bars: standard deviation across three cell pellet replicates.

FIG. 13 demonstrates HbA expression increases with VCN in erythroid cells obtained from β-thalassemia CD34⁺ cells. β-thalassemia CD34⁺ cells were transduced with LentiGlobin BB305 LVV at increasing MOI (2.5, 5, 10, 25, 25+SCTF), followed by erythroid differentiation for 14, 17, or 21 days. Freshly thawed and 48 h prestim only β-thalassemia CD34⁺ cells were used as controls in parallel cultures. VCN was measured at day 14 in erythroid culture. Triplicate cell pellets at the indicated days were analyzed by IE-HPLC. HbA peak assignment is based on AFSC hemoglobin control (FIG. 6A). HbA peak abundance is reported as % area of all hemoglobin peaks. Slope, γ-intercept, and β-squared values of linear regression are reported. Differences in slope and γ-intercept were not found to be significant (two-tailed p value >0.1).

FIGS. 14A-14D demonstrate transduction with LentiGlobin BB305 LVV rescues erythroid differentiation in β-thalassemia CD34⁺ cells after 14 days in culture. β-thalassemia CD34⁺ cells were either prestimulated for 48 hrs, or prestimulated and transduced with LentiGlobin BB305 LVV at MOI 25, followed by erythroid differentiation. A VCN of 0.62 was obtained from the cell culture at day 14. Cells were analyzed by FACS for size and DNA content at day 7 (FIG. 14A), day 11 (FIG. 14B), day 14 (FIG. 14C), and day 18 (FIG. 14D).

FIGS. 15A-15B demonstrate marker expression and cytospins confirm rescued enucleation in CD34⁺ cells transduced with LentiGlobin BB305 LVV. FIG. 15A shows cells corresponding to FIG. 14D (17 days of erythroid differentiation), were stained for viability, CD34, 45, 235a, 71, and DNA. CD235a/CD71 staining of the predominant CD34⁻/CD45⁻ population is shown. A β-fold increase in CD235a⁺/CD71⁻ cells was observed. Quadrant gates are drawn based on FMO controls. FIG. 15B shows cytospins confirm an increase in enucleated cells (arrows). Scale bar: 50 μm.

FIG. 16 demonstrates enucleation increases with increasing VCN in erythroid cells obtained from β-thalassemia CD34⁺ cells. β-thalassemia CD34⁺ cells were transduced with LentiGlobin BB305 LVV at increasing MOI (2.5, 5, 10, 25, 25+SCTF), followed by erythroid differentiation for 14, 17, or 21 days. Freshly thawed and 48 h prestim only β-thalassemia CD34⁺ cells were used as controls in parallel cultures. VCN was measured at day 14 in erythroid culture. Enucleated fraction of cells was quantified by FACS. Slope, γ-intercept, and β-squared values of linear regression are reported. Differences in slope between day 14 and day 17 had low significance (two-tailed p value=0.097). Differences in γ-intercepts between day 14 and day 17 were significant (p value=0.012).

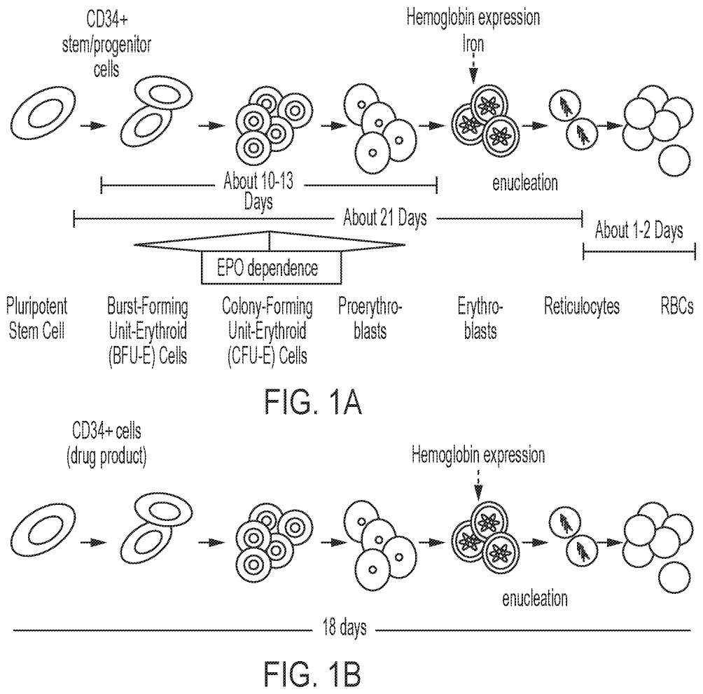

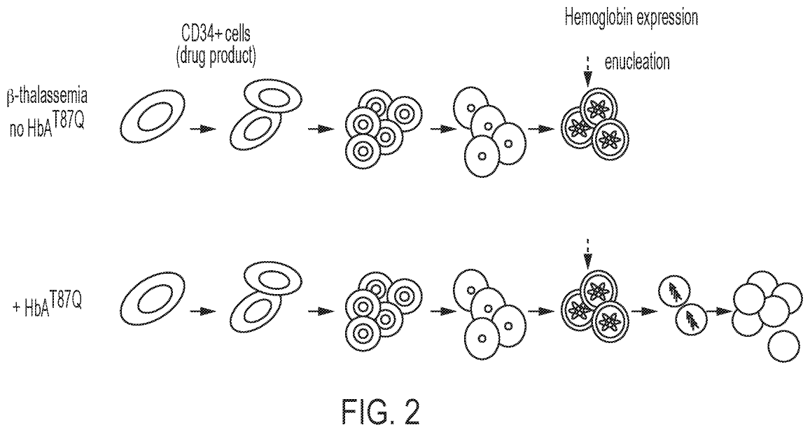

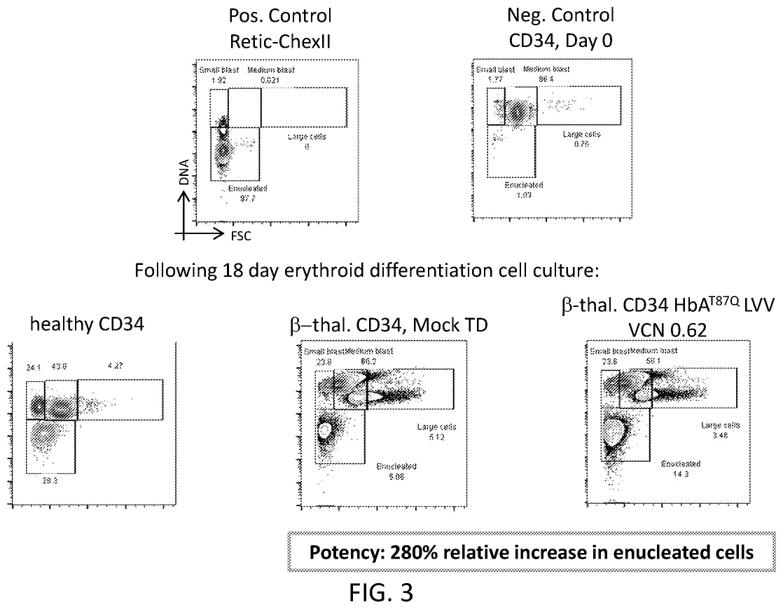

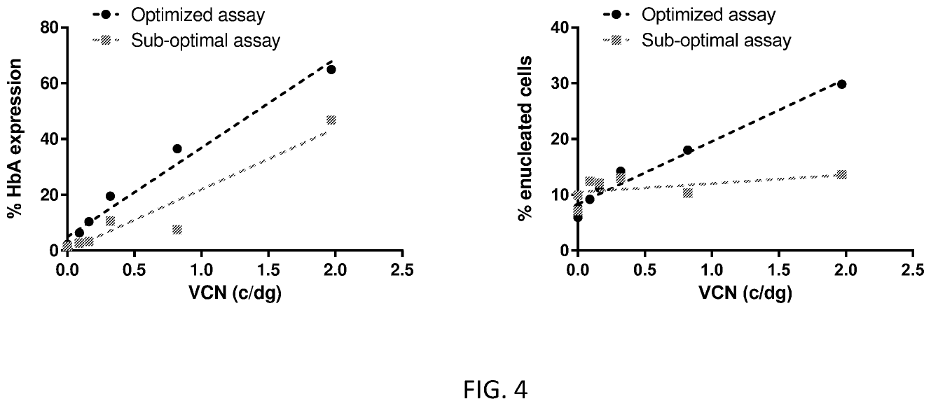

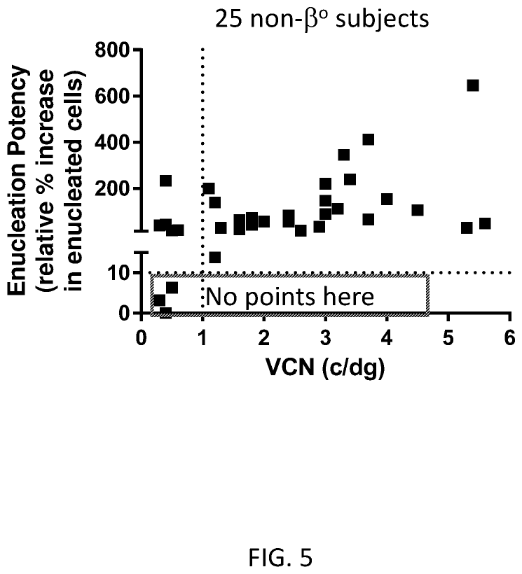

### DETAILED DESCRIPTION OF THE INVENTION

A robust and objective potency assay that can quantify the fold change in Hemoglobin A expression and/or the fold change in percent of enucleated reticulocytes in cells transduced with a lentiviral vector (LVV) comprising a polynucleotide encoding therapeutic globin compared to untransduced control cells is described herein. Moreover, this assay can be used to assess the correction of defects in erythroid differentiation and hemoglobin production associated with β-thalassemia. Disclosed herein are potency assays for a gene therapy treatment for β-thalassemia. Also disclosed herein are methods for measuring relative potency of a drug product.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements. The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. As used herein, the terms “include” and “comprise” are used synonymously. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., lentiviral vectors.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term “viral vector” may refer either to a virus or viral particle capable of transferring a nucleic acid into a cell or to the transferred nucleic acid itself. Viral vectors and transfer plasmids contain structural and/or functional genetic elements that are primarily derived from a virus. The term “lentiviral vector” refers to a retroviral vector or plasmid containing structural and functional genetic elements, or portions thereof, including LTRs that are primarily derived from a lentivirus. The terms “lentiviral vector” and “lentiviral expression vector” may be used to refer to lentiviral transfer plasmids and/or infectious lentiviral particles in particular embodiments. Where reference is made herein to elements such as cloning sites, promoters, regulatory elements, heterologous nucleic acids, etc., it is to be understood that the sequences of these elements are present in RNA form in the lentiviral particles contemplated herein and are present in DNA form in the DNA plasmids contemplated herein.

“Transfection” refers to the process of introducing naked DNA into cells by non-viral methods.

“Infection” refers to the process of introducing foreign DNA into cells using a viral vector. “Transduction” refers to the introduction of foreign DNA into a cell's genome using a viral vector.

“Vector copy number” or “VCN” refers to the number of copies of a vector, or portion thereof, in a cell's genome. The average VCN may be determined from a population of cells or from individual cell colonies.

“Transduction efficiency” refers to the percentage of cells transduced with at least one copy of a vector. For example if 1×10⁶ cells are exposed to a virus and 0.5×10⁶ cells are determined to have a least one copy of a virus in their genome, then the transduction efficiency is 50%.

The term “globin” as used herein refers to proteins or protein subunits that are capable of covalently or noncovalently binding a heme moiety, and can therefore transport or store oxygen. Subunits of vertebrate and invertebrate hemoglobins, vertebrate and invertebrate myoglobins or mutants thereof are included by the term globin. The term excludes hemocyanins. Examples of globins include α-globin or variants thereof, β-globin or variants thereof, a γ-globin or variants thereof, and δ-globin or variants thereof.

As used herein, the term “thalassemia” refers to a hereditary disorder characterized by defective production of hemoglobin. Examples of thalassemias include α- and β-thalassemia. β-thalassemias are caused by a mutation in the β-globin chain, and can occur in a major or minor form. Nearly 400 mutations in the β-globin gene have been found to cause β-thalassemia. Most of the mutations involve a change in a single DNA building block (nucleotide) within or near the β-globin gene. Other mutations insert or delete a small number of nucleotides in the β-globin gene. As noted above, β-globin gene mutations that decrease β-globin production result in a type of the condition called beta-plus (β⁺) thalassemia. Mutations that prevent cells from producing any β-globin result in beta-zero (β⁰) thalassemia. In the major form of β-thalassemia, children are normal at birth, but develop anemia during the first year of life. The minor form of β-thalassemia produces small red blood cells. Thalassemia minor occurs if you receive the defective gene from only one parent. Persons with this form of the disorder are carriers of the disease and usually do not have symptoms.

Additional definitions are set forth throughout this disclosure.

In the following description, certain specific details are set forth in order to provide a thorough understanding of various illustrative embodiments of the invention contemplated herein. However, one skilled in the art will understand that particular illustrative embodiments may be practiced without these details.

### Potency Assays

Disclosed herein are potency assays for a gene therapy treatment for β-thalassemia. In some embodiments a potency assay comprises transducing a sample of hematopoietic stem or progenitor cells from a subject (e.g., a subject who has β-thalassemia) with a vector (e.g., a lentiviral vector) comprising a polynucleotide encoding a globin; erythroid differentiating the transduced hematopoietic stem or progenitor cells; erythroid differentiating a sample of untransduced hematopoietic stem or progenitor cells from the subject having β-thalassemia; measuring fold change in Hemoglobin A expression in the transduced and the untransduced erythroid cell samples; and/or measuring fold change in enucleated reticulocytes in the transduced and the untransduced erythroid cell samples. The potency of the gene therapy may be assessed as the fold change in HbA expression and/or fold change in percent enucleated reticulocytes in the transduced compared to the untransduced erythroid cell samples.

In some embodiments, a potency assay for a gene therapy treatment for 1-thalassemia comprises transducing a first sample of hematopoietic stem or progenitor cells from a subject having β-thalassemia with a lentiviral vector comprising a polynucleotide encoding a globin; performing erythroid differentiation of the first sample of hematopoietic stem or progenitor cells; performing erythroid differentiation of a second sample of untransduced hematopoietic stem or progenitor cells from the subject having β-thalassemia; measuring fold change in Hemoglobin A expression in the transduced and the untransduced erythroid cell samples; and measuring fold change in enucleated reticulocytes in the transduced and the untransduced erythroid cell samples, wherein the potency of the gene therapy is assessed as the fold change in HbA expression and/or fold change in percent enucleated reticulocytes, in the first sample compared to the second sample.

In particular aspects, the method comprises obtaining a sample of hematopoietic stem or progenitor cells from a subject that has β-thalassemia. Suitable methods for obtaining hematopoietic stem or progenitor cells from a subject include apheresis.

In some aspects hematopoietic stem or progenitor cells are selected from the group consisting of CD34⁺ cells, CD133⁺ cells, CD34⁺CD133⁺ cells, CD34⁺CD38^{Lo}CD90⁺CD45RA⁻ cells, and combinations thereof. In certain aspects, the hematopoietic stem or progenitor cells include CD34⁺ cells. In certain aspects, the hematopoietic stem or progenitor cells include CD133⁺ cells. In certain aspects, the hematopoietic stem or progenitor cells include CD34⁺CD133⁺ cells. In certain aspects, the hematopoietic stem or progenitor cells include CD34⁺CD38^{Lo}CD90⁺CD45RA⁻ cells.

In some aspects, the hematopoietic stem or progenitor cells comprise a pair of β-globin alleles selected from the group consisting of β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺. In certain aspects, the hematopoietic stem or progenitor cells comprise a pair of β-globin alleles that are β^{E}/β⁰. In certain aspects, the hematopoietic stem or progenitor cells comprise a pair of β-globin alleles that are β^{C}/β⁰. In certain aspects, the hematopoietic stem or progenitor cells comprise a pair of β-globin alleles that are β⁰/β⁰. In certain aspects, the hematopoietic stem or progenitor cells comprise a pair of β-globin alleles that are β^{C}/β^{C}. In certain aspects, the hematopoietic stem or progenitor cells comprise a pair of β-globin alleles that are β^{E}/β^{E}. In certain aspects, the hematopoietic stem or progenitor cells comprise a pair of β-globin alleles that are β^{E}/β⁺. In certain aspects, the hematopoietic stem or progenitor cells comprise a pair of β-globin alleles that are β^{C}/β^{E}. In certain aspects, the hematopoietic stem or progenitor cells comprise a pair of β-globin alleles that are β^{C}/β⁺. In certain aspects, the hematopoietic stem or progenitor cells comprise a pair of β-globin alleles that are β⁰/β⁺. In certain aspects, the hematopoietic stem or progenitor cells comprise a pair of β-globin alleles that are β^{E}/β^{E}. In certain aspects, the hematopoietic stem or progenitor cells comprise a pair of β-globin alleles that are β⁺/β⁺.

In some embodiments, the hematopoietic stem or progenitor cells are transduced with a vector (e.g., a lentiviral vector) comprising a polynucleotide encoding a globin. In some aspects, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In certain aspects, the globin is a human β-globin protein. In certain aspects, the globin is a human δ-globin protein. In certain aspects, the globin is an anti-sickling globin protein. In certain aspects, the globin is a human γ-globin protein. In certain aspects, the globin is a human β^{A-T87Q}-globin protein. In certain aspects, the globin is a human β^{A-G16D/E22A/T87Q}-globin protein. In certain aspects, the globin is a human β^{A-T87Q/K95E/K120E}-globin protein.

In some embodiments, the vector is a lentiviral vector. In some aspects the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a GLOBE vector, a G-GLOBE vector, a βAS3-FB vector, or a derivative thereof. In some aspects, the lentiviral vector is an AnkT9W vector or a derivative thereof. In some aspects, the lentiviral vector is a T9Ank2W vector or a derivative thereof. In some aspects, the lentiviral vector is a TNS9 vector or a derivative thereof. In some aspects, the lentiviral vector is a TNS9.3 vector or a derivative thereof. In some aspects, the lentiviral vector is a TNS9.3.55 vector or a derivative thereof. In some aspects, the lentiviral vector is a lentiglobin HPV569 vector or a derivative thereof. In some aspects, the lentiviral vector is a lentiglobin BB305 vector or a derivative thereof. In some aspects, the lentiviral vector is a BG-1 vector or a derivative thereof. In some aspects, the lentiviral vector is a BGM-1 vector or a derivative thereof. In some aspects, the lentiviral vector is a GLOBE vector or a derivative thereof. In some aspects, the lentiviral vector is a G-GLOBE vector or a derivative thereof. In some aspects, the lentiviral vector is a βAS3-FB vector or a derivative thereof.

In some aspects, the transduced hematopoietic stem or progenitor cells are erythroid differentiated. In some aspects, the erythroid differentiation method comprises a two-stage culture. The two-stage erythroid differentiation of the transduced hematopoietic stem or progenitor cells occurs for a period of at least 14, at least 15, at least 16, at least 17, at least 18, at least 19, at least 20, at least 21, at least 22, at least 23, at least 24, or at least 25 days. In certain aspects, the first phase of erythroid differentiation occurs for a period of 1 to 10 days, or preferably for a period of 7 days. In certain aspects, the second phase of erythroid differentiation occurs for a period of 1 to 15 days, or preferably for a period of 7 days. In some embodiments, the first phase of erythroid differentiation occurs from day 1 to day 7 and the second phase of erythroid differentiation occurs from day 7 to days 14-17, preferably day 17, of the differentiation method.

In some embodiments, the culturing of the transduced hematopoietic stem or progenitor cells in the first phase of erythroid differentiation occurs in a first medium and the culturing of the transduced hematopoietic stem or progenitor cells in the second phase of erythroid differentiation occurs in a second medium. For example, the transduced hematopoietic stem or progenitor cells may be cultured in a first medium for days 1-7 of erythroid differentiation, and at day 7 the cells are moved to a second medium and then cultured in the second medium for day 7 to days 14-17, preferably day 17, of erythroid differentiation.

In some embodiments, the fold change in Hemoglobin A expression is measured for transduced erythroid cell samples. In some embodiments, the fold change in Hemoglobin A expression is measured for untransduced erythroid cell samples. In some aspects, the fold change in Hemoglobin A (HbA) expression is measured using high-performance liquid chromatography (HPLC) (e.g., ion-exchange HPLC). In some aspects, the potency of a gene therapy is assessed as the fold change in HbA expression in transduced compared to untransduced erythroid cell samples.

In some embodiments, the fold change in enucleated reticulocytes is measured for transduced erythroid cell samples. In some embodiments, the fold change in enucleated reticulocytes is measured for untransduced erythroid cell samples. In some aspects, the fold change in enucleated reticulocytes is measured using flow cytometry (e.g., fluorescence-activated cell sorting (FACS)). In some aspects, the potency of a gene therapy is assessed as the fold change in enucleated reticulocytes in transduced compared to untransduced erythroid cell samples.

In some aspects, the potency of a gene therapy is assessed as the measured fold change in Hemoglobin A expression and the measured fold change in enucleated reticulocytes for transduced erythroid cell samples compared to untransduced erythroid cell samples.

### Methods for Measuring Potency of a Drug Product

Also disclosed herein are methods for measuring relative potency of a drug product. In some aspects, the methods comprise quantifying the fold change in Hemoglobin A (HbA) expression in transduced and untransduced erythroid cells. In some aspects, the methods comprise quantifying the fold change in enucleated reticulocytes in transduced and untransduced cells. In certain aspects, the methods comprise quantifying the fold change in Hemoglobin A (HbA) expression and the fold change in enucleated reticulocytes in transduced and untransduced cells.

In some embodiments, the transduced cells are transduced erythroid cells. The transduced erythroid cells may be obtained by transducing hematopoietic stem or progenitor cells with a viral vector (e.g., a lentiviral vector) comprising a polynucleotide encoding a globin. In some aspects, the hematopoietic stem or progenitor cells comprise CD34⁺ cells, CD133⁺ cells, or CD34⁺CD38^{Lo}CD90⁺CD45RA⁻ cells. In some aspects a lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a GLOBE vector, a G-GLOBE vector, a βAS3-FB vector, or a derivative thereof. In some aspects, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein.

In some embodiments, the hematopoietic stem or progenitor cells are obtained from a patient or subject having β-thalassemia (e.g., β-thalassemia major).

In some embodiments, the fold change in Hemoglobin A expression is measured using HPLC (e.g., ion-exchange HPLC). In some embodiments, the fold change in enucleated reticulocytes in measuring using flow cytometery (e.g., FACS).

In some embodiments, the hematopoietic stem or progenitor cells transduced with the lentiviral vector are differentiated using a two-phase erythroid differentiation protocol before the fold change in Hemoglobin A expression and/or the fold change in enucleated reticulocytes is measured. In some aspects, the erythroid differentiation protocol occurs over a period of 14 to 17 days.

All publications, patent applications, and issued patents cited in this specification are herein incorporated by reference as if each individual publication, patent application, or issued patent were specifically and individually indicated to be incorporated by reference.

The following examples are provided by way of illustration only and not by way of limitation. Those of skill in the art will readily recognize a variety of noncritical parameters that could be changed or modified in particular embodiments to yield essentially similar results.

### EXEMPLIFICATION

### Summary

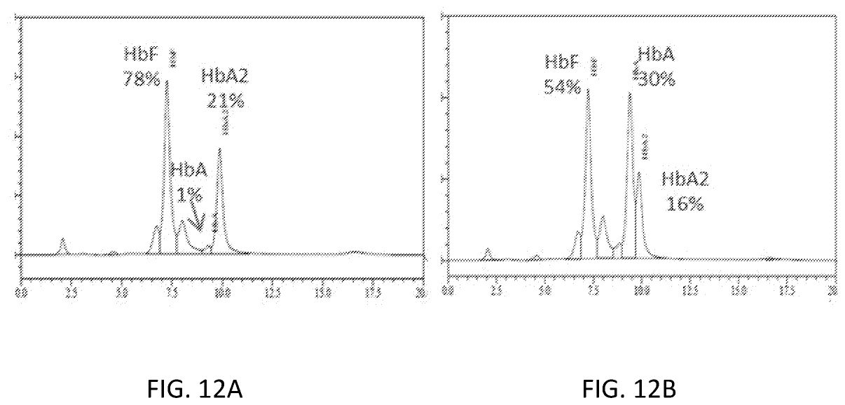

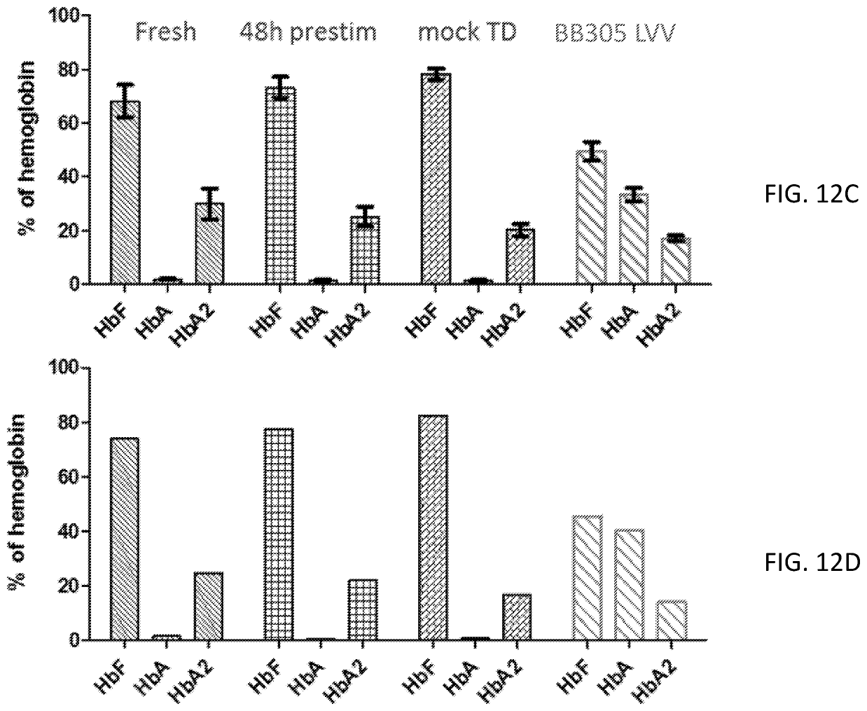

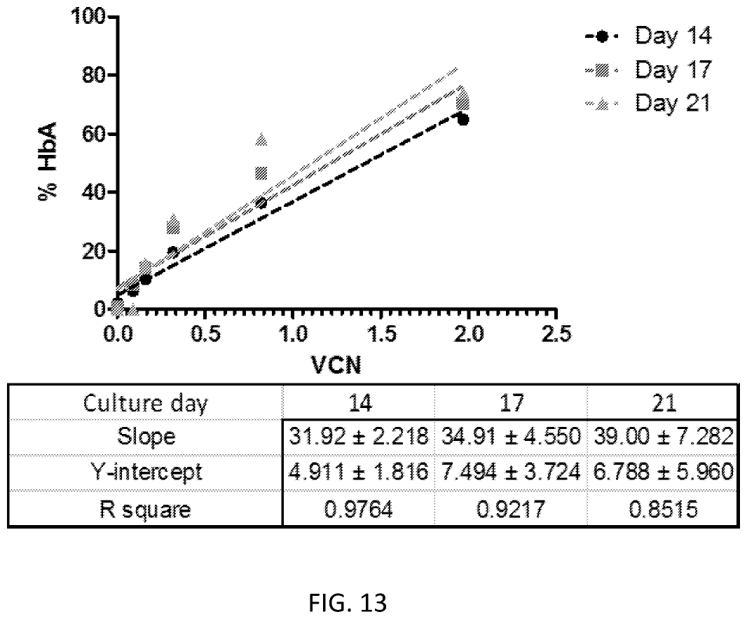

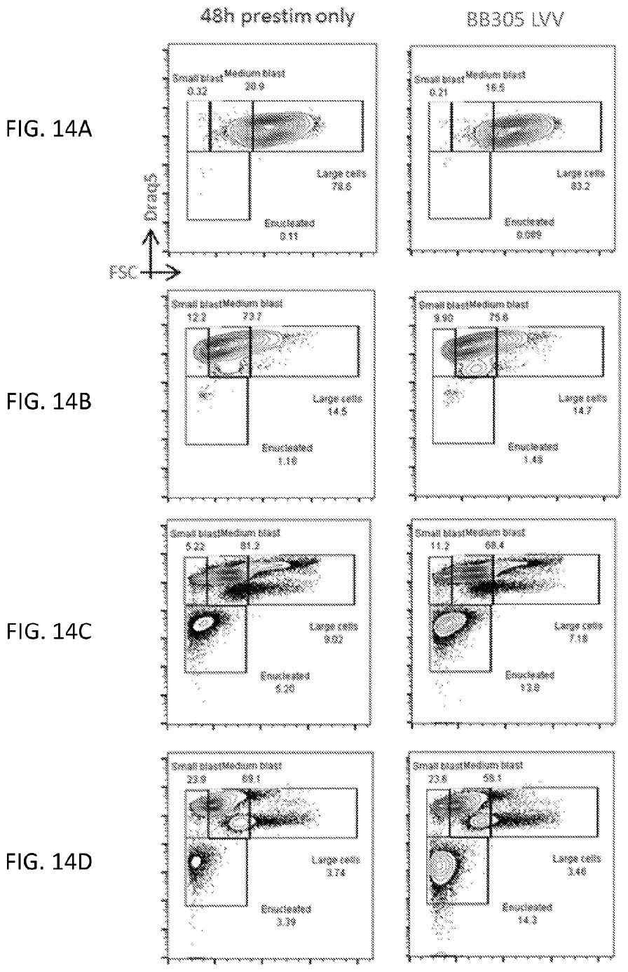

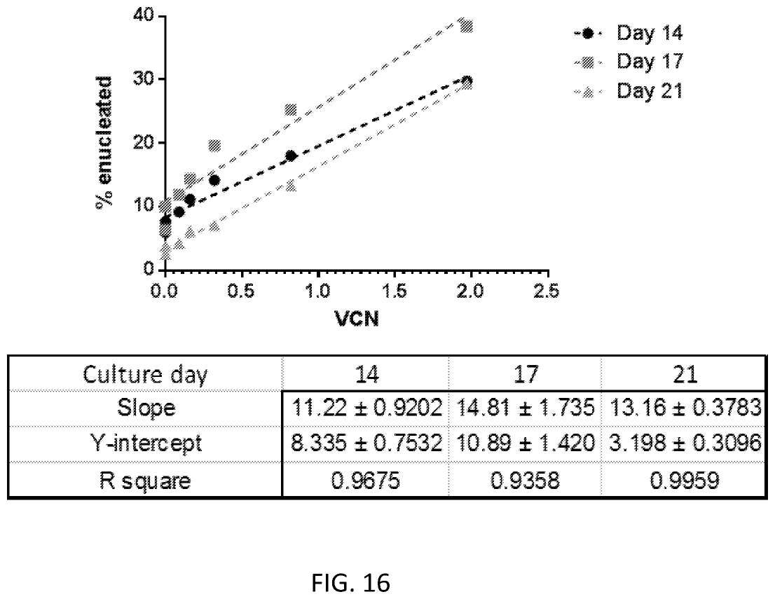

An assay has been developed to evaluate the potency of a lentiviral vector (LVV) encoding a globin including, but not limited to, β-globin, or an anti-sickling β-globin (e.g., β-globinAT87Q) in rescuing erythropoiesis in a Drug Product manufactured from CD34⁺hematopoietic stem and progenitor cells (HSPCs) obtained from patients with β-thalassemia. In this assay, HPSCs transduced with LentiGlobin BB305 LVV and untransduced HSPCs from day 2 of manufacturing were erythroid differentiated in two-stage culture for 14-17 days. The fold increase in expression of Hemoglobin A (HbA) from transduced and untransduced HSPCs was quantified by IE-HPLC and the fold increase in enucleated cell abundance (reticulocytes and erythrocytes) was quantified by FACS. At day 18 of culture, an 86-fold increase in HbA abundance and a 4.2-fold increase in enucleated cells were observed (FIG. 12D, FIG. 14D). Potency readouts at day 14 of erythroid differentiation showed a smaller but still substantial effect, an 18-fold increase in HbA abundance, 2.6-fold increase in enucleated cells (FIG. 12C, FIG. 14C). Transduction with LentiGlobin BB305 LVV was shown to rescue erythroid differentiation in β-thalassemia CD34⁺ cells, with a proportional relationship to vector copy number (VCN), i.e., potency increased with increasing VCN (FIG. 13, FIG. 16).

### Results

Determination of Hemoglobin Composition by IE-HPLC

A cell culture method to evaluate the potency of a LVV encoding a therapeutic globin, e.g., β-globin^{T87Q} should mimic endogenous globin chain selection programs. CD34⁺ cells from healthy adults should primarily express HbA upon differentiation. If globin switching is perturbed and non-physiological globin chains, such as HbF, are expressed, that alone would ameliorate β-thalassemia diserythropoiesis, masking any potency from expression of β-globin^{T87Q}.

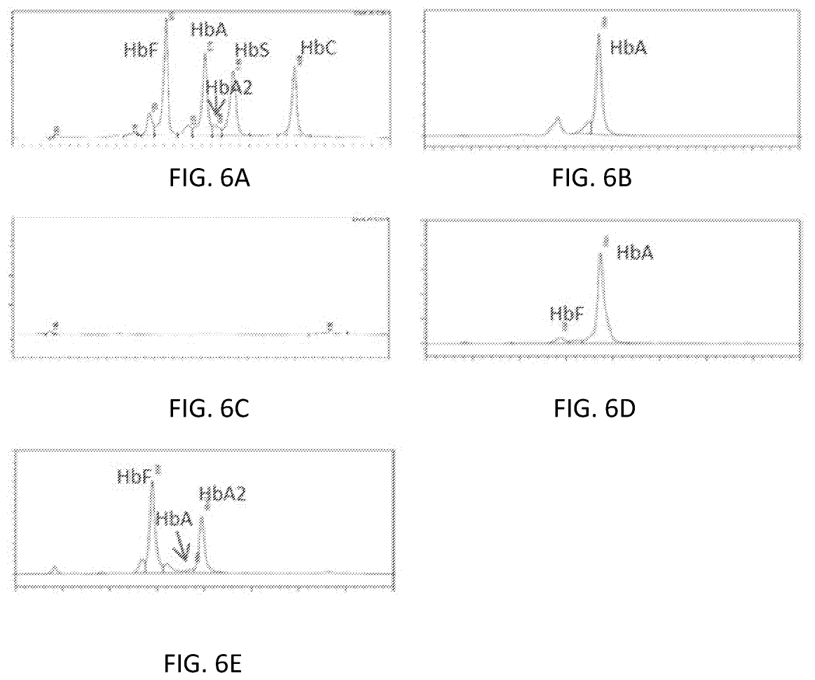

To quantitatively evaluate a composition of expressed hemoglobins, an ion-exchange method was developed. Unlike reverse-phase HPLC, the hemoglobin chains are not denatured (FIG. 6A), simplifying quantitative hemoglobin composition analysis in β-thalassemia, where excess α-chain expression convolutes analysis by reverse-phase HPLC (FIG. 6F). Because absorbance with bound heme is measured, only intact hemoglobins are detected, leading to absence of signal in undifferentiated CD34⁺ cells (FIG. 6C). Using IE-HPLC, hemoglobin composition was evaluated for the differentiated cells. The differentiated cells gave consistently low HbF expression (FIG. 6D) indicating the cells are similar to those found in adult blood (FIG. 6B).

Linearity and Precision of IE-HPLC

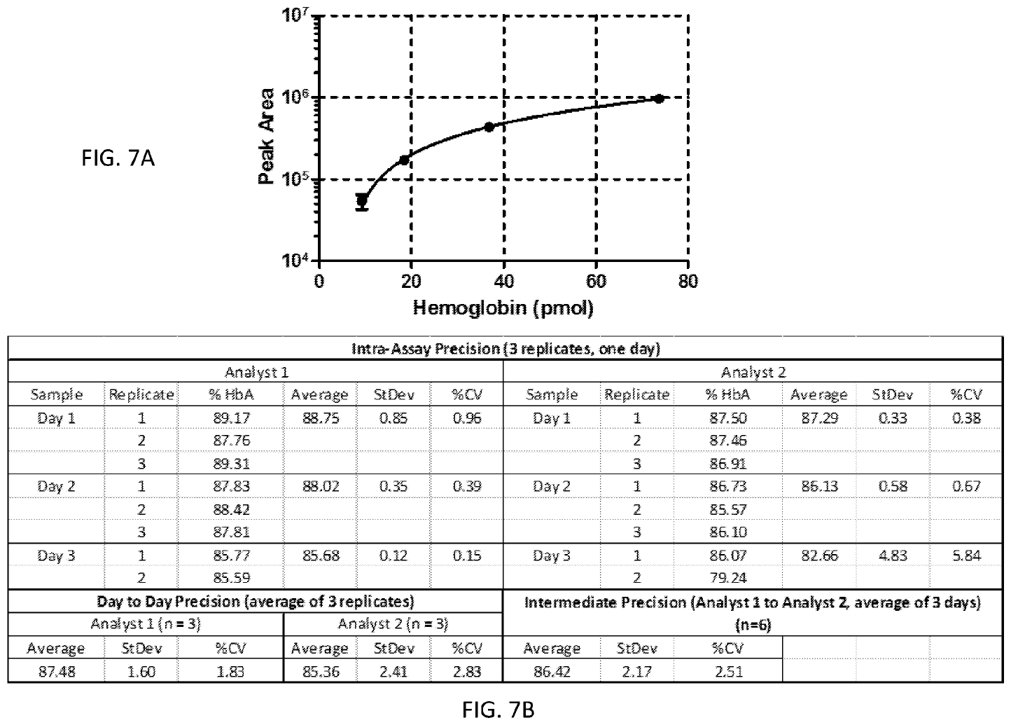

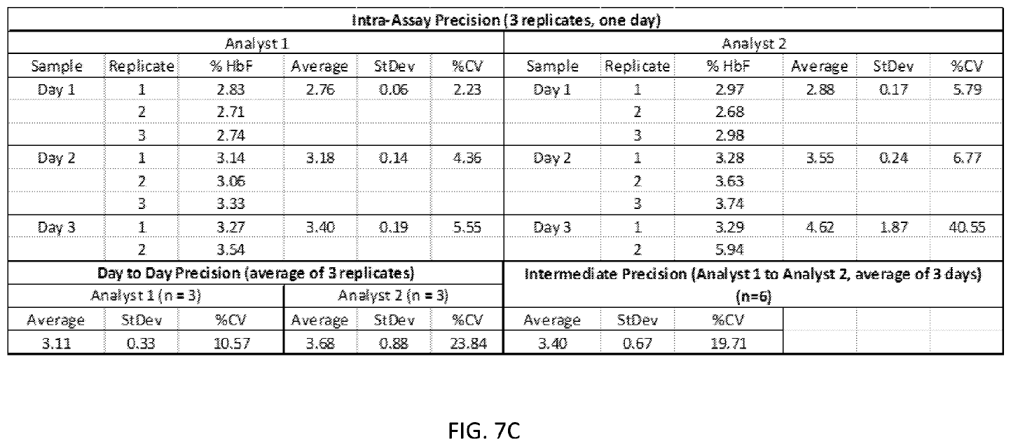

Linearity and precision of the IE-HPLC method were measured. Using serial dilutions of the HbA/HbA2 hemoglobin standard the method was highly linear (FIG. 7A, R²=0.999). Precision of lysing 1×10⁶ cells and analyzing 6×10⁴ cells equivalents had a maximum % CV of 5.84 for HbA (86% of all hemoglobins in sample, FIG. 7B) and 40.55% for HbF (3.4% of all hemoglobins in sample, FIG. 7C).

Identification of Enucleated Cells by FACS

Erythroid differentiation of CD34⁺ cells proceeds through distinct steps. Progenitor cells give rise to prepro-erythroblasts that are larger in size and begin to express CD71 while CD34 and CD45 expression declines. As pro-erythroblasts and basophilic erythroblasts form, CD71 expression peaks, CD235a (glycophorin A) begins to be expressed, and cell size declines. Approaching polychromatic and orthochromatic erythroblasts, cell size declines, CD235a expression peaks, CD71 expression declines, and hemoglobin expression ramps up. Cells then enucleate to form reticulocytes (rRNAL^{lo}, CD71^{lo}, DNA⁻, CD235a⁺, CD34⁻, CD45⁻) that mature to erythrocytes (rRNA⁻, CD71⁻, DNA⁻, CD235a⁺, CD34⁻, CD45⁻).

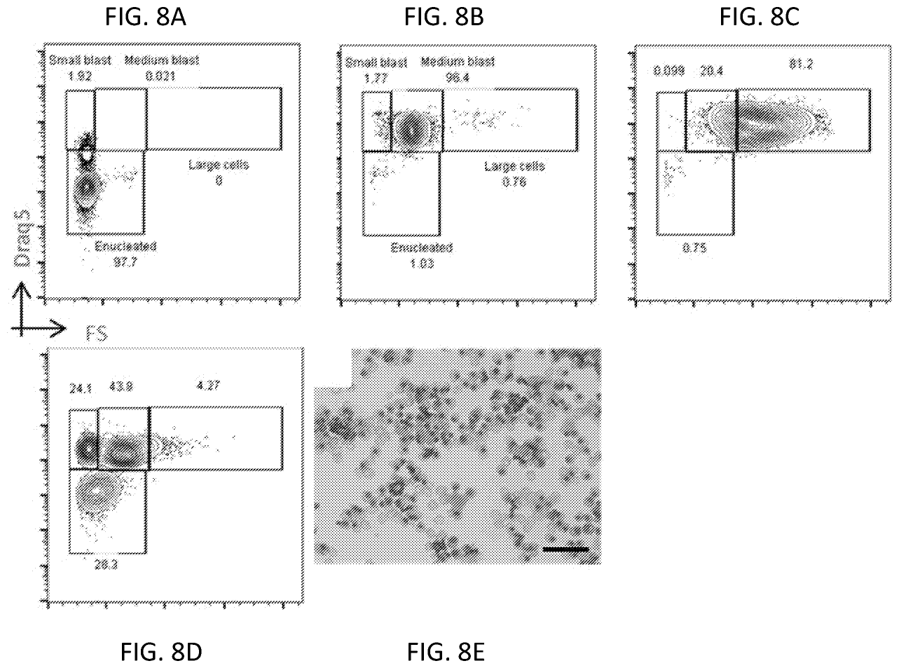

An assay was developed to track the changes in erythroid differentiation culture simply by size and DNA content. Undifferentiated CD34⁺ cells become larger than early erythroblasts during 7 days in culture (FIGS. 8B-8C), and by day 14 resolve to two populations of small erythroblasts and one population of reticulocytes/erythrocytes (FIG. 8D), resembling the 1-2% reticulocyte/erythrocyte control ReticChexII (FIG. 8A).

Precision of FACS-Based Enucleation Assay

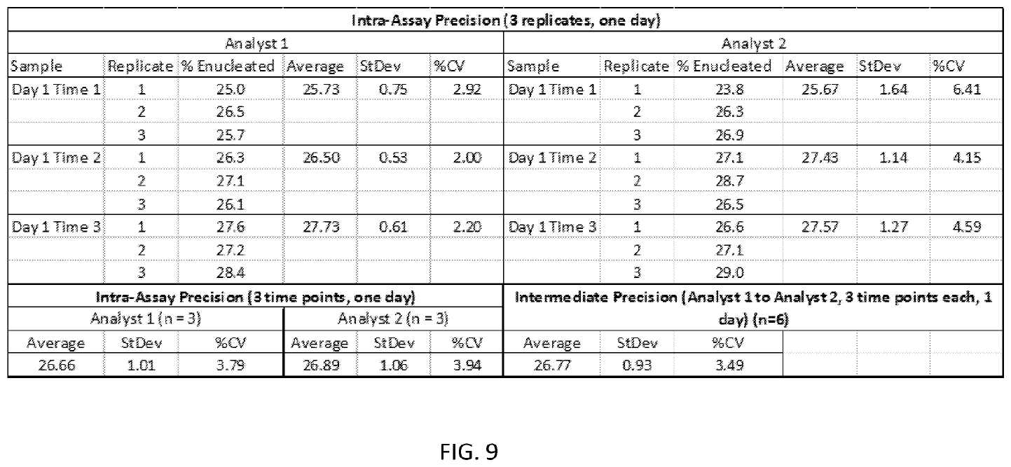

Intra-assay and intermediate precision of measuring enucleation was tested using a single batch of healthy erythroid-differentiated cells (FIG. 9). Maximum intra-assay CV from 3 replicates was 6.41%. Maximum intra-day and analyst-to-analyst CV was 3.94%.

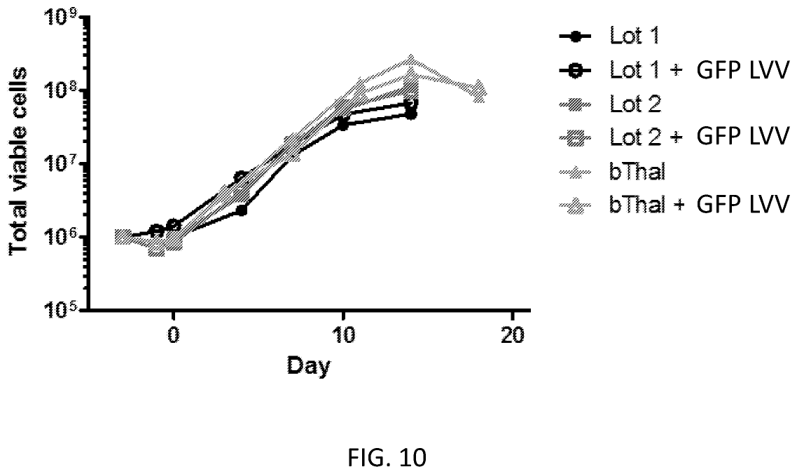

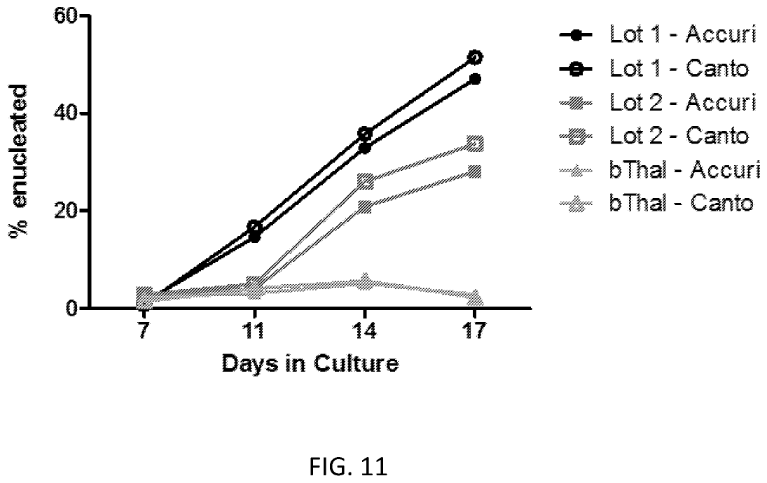

Healthy and β-thalassemia cells from different cell lots had similar growth kinetics, peaking by day 14 in culture (FIG. 10). Cell growth was unaffected by transduction with a LVV encoding GFP. Enucleation was low and variable at day 11 with indistinguishable abundances of reticulocytes between healthy lot 2 and β-thalassemia cells (FIG. 11). Enucleation increased by day 14, with a clear difference between healthy and β-thalassemia cell lots. By day 17, enucleation increased further in healthy samples and decreased in the β-thalassemia samples. The preferred culture duration for the potency assay is therefore 14-17 days. Comparable enucleation values and trends were obtained from the same samples using both BD-Canto and BD-Accuri flow cytometers.

Rescue of HbA Expression and Enucleation in β-Thalassemia CD34 Cells Transduced with LentiGlobin BB305 LVV is Linearly Dependent on VCN

The potency of LentiGlobin BB305 LVV in increasing Hemoglobin A (HbA) expression was evaluated in β-thalassemia CD34⁺ cells transduced at a vector copy number (VCN) of 0.62. Untransduced cells had a negligible amount of HbA that increased to 33% of all hemoglobins by day 14 and 40% by day 18 of erythroid differentiation (FIG. 12). Rescue was linearly dependent with VCN (FIG. 13), with insignificant differences in slope between readouts at day 14, 17, or 21.

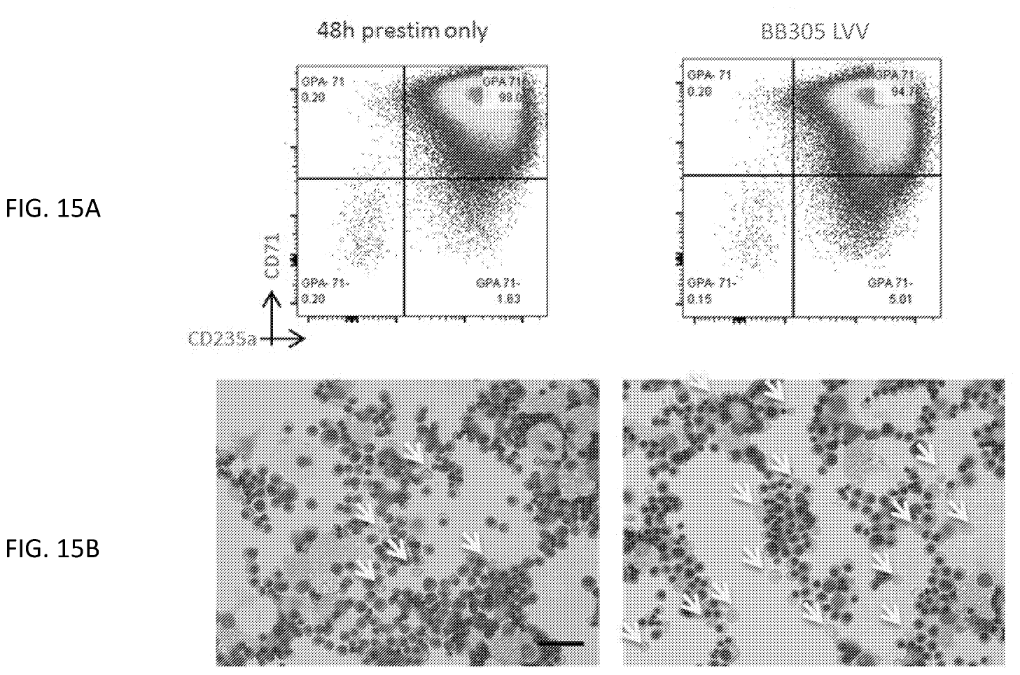

The potency of LentiGlobin BB305 LVV in increasing the abundance of differentiated, enucleated reticulocytes was evaluated in β-thalassemia CD34⁺ cells transduced at VCN of 0.62. No difference was observed at day 7 or 11, consistent with the observed incomplete progression through differentiation (FIGS. 14A-14B). However, by day 14, the transduced cells had a 2.6-fold increase in enucleation that increased to 4.2-fold by day 18 (FIGS. 14C-14D). Consistent with these observations, the abundance of CD235⁺/CD71⁻ cells increased 3.1-fold by day 18, and more enucleated cells were observed by cytospins (FIG. 15). Enucleation increased with increasing VCN (FIG. 16), with a higher potency (slope) observed at day 17 than day 14. Culture to 21 days did not further increase potency.

### Methods

Erythroid Differentiation Culture

CD34⁺ cells (0.5-2×10⁶) were plated in media A (IMDM, 20% FBS, rhSCF (20 ng/mL), rhIL3 (1 ng/mL), rhEPO (2 U/mL)) at 1×10⁶ cells/mL in non-TC treated 12 well plate at 1-2 mL/well. Cells were incubated at 37° C. 5% CO₂. At β-4 days in culture, cell count, viability, and average size were obtained on the ViCell XR (Beckman Coulter). 1-2×10⁶ cells were removed, diluted to 5×10⁵ cells/mL with fresh media A, and plated in non-TC treated 12 well plate at 1-2 mL/well. At 7 days in culture, cells were collected by centrifugation (500×g, 5 min) and resuspended in 10 mL IMDM. Cell count, viability, and average size were obtained on the ViCell XR. β-6×10⁶ cells were collected by centrifugation (500×g, 5 min) and resuspended in media B (IMDM, 20% FBS, rhEPO (2 U/mL), human apo-transferrin (0.2 mg/mL)) at 5×10⁵ cells/mL in non-TC treated 6 well plate at 2-3 mL/well. At 10-11 days in culture, cell count, viability, and average size were obtained on the ViCell XR. 9-18×10⁶ cells were removed, diluted to 5×10⁵ cells/mL with fresh media B, and plated in non-TC treated 6 well plate at 2-3 mL/well. For cultures longer than 14 days, addition of up to 50% fresh media B continued every β-4 days, maintaining cell density of 1×10⁶ cells/mL.

Separation and Identification of Hemoglobins by Ion-Exchange HPLC

Cultured cells were resuspended in PBS containing 2% FBS, aliquoted at 1×10⁶ cells per tube, centrifuged (500×g, 5 min), and supernatant was aspirated. Cell pellets were frozen at −80° C. until analysis. Frozen pellets were resuspended in lysis buffer (100 uL), incubated 10 min at room temperature, vortexed, and diluted with water (400 uL). Cell debris was removed by centrifugation (20,000×g, 30 min, 4° C.), and 30 uL of supernatant was used for each HPLC analysis. Hemoglobins were resolved using Polycat A column (200×2.1 mm, 5 um, 1000 Å) on a Shimadzu UFLC system equipped with LC20AD pumps, SIL20ACHT autosampler, and SPD20A detector set to 418 nm. Mobile phase A: 40 mM Tris, 3 mM KCN, pH 6.5. Mobile phase B: 0.2M NaCl, 40 mM Tris, 3 mM KCN, pH 6.5. Flow rate: 0.3 mL/min. Gradient:

| Time (mm:ss) | % B |
|---|---|
| 00:01 | 2 |
| 00:30 | 20 |
| 02:00 | 20 |
| 06:00 | 60 |
| 08:00 | 60 |
| 12:00 | 100 |
| 12:30 | 100 |

Water blanks were injected prior to data collection and in-between samples. Retention times of HbF, HbA, and HbA2 were determined from the AFSC hemoglobin control (diluted 1:1000 in water, 10 uL injection). To determine relative abundance of each peak, the integrated area of each peak was divided by the total integrated area of all hemoglobin peaks. To determine linearity, HbA/HbA2 hemoglobin control (4 uL) was dissolved in water (996 uL) and serially-diluted 2-fold 4 times.

FACS Analysis of Erythroid Differentiation

For nuclear staining, cultured cells were resuspended in PBS containing FBS (2%), aliquoted at 5×10⁵ cells per tube, centrifuged (500×g, 5 min), and supernatant was aspirated. Cells were resuspended in 400 uL staining buffer (PBS, 2% FBS, 1:5000 Draq5), incubated 10 min, and 200 uL of each sample was analyzed on a BD-Accuri flow cytometer. Cells were separated from debris using FSC/SSC gates, and enucleated cells along with erythroblast subpopulations were identified using FSC/Draq5 gates. Spherotech 6-peak validation beads were used as a system suitability control. ReticChexII were used as a positive control for enucleated cells, diluting 1:10,000 in staining buffer. For analysis using the BD-Canto flow cytometer, 5×10⁵ cells were resuspended in Live/Dead Aqua (1:1000), incubated 10 min, and pelleted by centrifugation (500×g, 5 min). Supernatant was removed and cells were resuspended in 400 uL staining buffer (PBS, 2% FBS, 1:2500 Draq5), incubated 10 min, and analyzed. Cells were separated from debris using FSC/SSC gates, live cells were gated based on Aqua (AmCyan), and enucleated cells along with erythroblast subpopulations were identified using FSC/Draq5(APC) gates. For CD235/CD71 staining, cultured cells (1×10⁶) were washed once with PBS and stained for 30 min at 4° C. in 100 uL FACS buffer (PBS, 2% human serum albumin) containing CD45-BV510 (5 uL), CD34-A700 (5 uL), CD71-APC (5 uL), CD235α-PE (2.5 uL), Live/Dead fixable far red (1/1000). Stained cells were diluted with 100 uL FACS buffer, collected by centrifugation, and stained for 30 min at room temperature in 100 uL PBS with DyeCycle violet (1/4000). Analysis was performed with a BD-Fortessa flow cytometer within 1 hour of DyeCycle staining. Gates were drawn using compensated parameters and Flowjo software, with undifferentiated CD34⁺ cells and ReticChexII as controls.

Identification of Enucleated Red Blood Cells by Cytospin

Cultured cells (1×10⁶) were resuspended in sterile filtered PBS containing 10% FBS (200 uL), loaded into cytofunnels, and cytospun at 800 rpm for 5 min with medium acceleration. Cytospin slides were dried overnight, stained in Wright-Giemsa stain for 3 min, destained in water for 7 min, and thoroughly rinsed. After drying overnight, slides were imaged at 40× on a Nikon Eclispe TS100 microscope equipped with brightfield illumination and Nikon DS-Fi2 camera.

What is claimed is:

1. A potency assay for a gene therapy treatment for β-thalassemia comprising:
a) transducing a sample of hematopoietic stem or progenitor cells from a subject having β-thalassemia with a lentiviral vector comprising a polynucleotide encoding a globin;
b) erythroid-differentiating the transduced hematopoietic stem or progenitor cells;
c) erythroid-differentiating a sample of untransduced hematopoietic stem or progenitor cells from the subject having β-thalassemia;
d) measuring fold change in Hemoglobin A expression in the transduced and the untransduced erythroid cell samples; and
e) measuring fold change in enucleated reticulocytes in the transduced and the untransduced erythroid cell samples,
wherein the potency of the gene therapy is assessed as the fold change in HbA expression and/or fold change in percent enucleated reticulocytes, in the transduced compared to the untransduced erythroid cell samples.

2. A potency assay for a gene therapy treatment for β-thalassemia comprising:
a) transducing a sample of hematopoietic stem or progenitor cells from a subject having β-thalassemia with a lentiviral vector comprising a polynucleotide encoding a globin;
b) erythroid-differentiating the transduced hematopoietic stem or progenitor cells;
c) erythroid-differentiating a sample of untransduced hematopoietic stem or progenitor cells from the subject having β-thalassemia; and
d) measuring fold change in Hemoglobin A expression in the transduced and the untransduced erythroid cell samples,
wherein the potency of the gene therapy is assessed as the fold change in HbA expression in the transduced compared to the untransduced erythroid cell samples.

3. A potency assay for a gene therapy treatment for β-thalassemia comprising:
a) transducing a sample of hematopoietic stem or progenitor cells from a subject having β-thalassemia with a lentiviral vector comprising a polynucleotide encoding a globin;
b) erythroid-differentiating the transduced hematopoietic stem or progenitor cells;
c) erythroid-differentiating a sample of untransduced hematopoietic stem or progenitor cells from the subject having β-thalassemia; and
d) measuring fold change in enucleated reticulocytes in the transduced and the untransduced erythroid cell samples,
wherein the potency of the gene therapy is assessed as the fold change in percent enucleated reticulocytes in the transduced compared to the untransduced erythroid cell samples.

4. The potency assay of claim 1, further comprising obtaining the hematopoietic stem or progenitor cells from the subject that has β-thalassemia.

5. The potency assay of any one of claims 1 to 4, wherein the hematopoietic stem or progenitor cells comprise CD34+ cells.

6. The potency assay of any one of claims 1 to 5, wherein the hematopoietic stem or progenitor cells comprise CD133⁺ cells.

7. The potency assay of any one of claims 1 to 6, wherein the hematopoietic stem or progenitor cells comprise CD34⁺CD38^{Lo}CD90⁺CD45RA⁻ cells.

8. The potency assay of any one of claims 1 to 7, wherein the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

9. The potency assay of any one of claims 1 to 8, wherein the globin is a human β-globin, an anti-sickling globin, a human β^{A-T87Q}-globin, a human β^{A-G16D/E22A/T87Q_} globin, or a human β^{A-T87Q/K95E/K120E}-globin protein.

10. The potency assay of any one of claims 1 to 9, wherein the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a GLOBE vector, a G-GLOBE vector, a βAS3-FB vector, or a derivative thereof.

11. The potency assay of any one of claims 1 to 10, wherein the erythroid differentiation method comprises a two-stage culture.

12. The potency assay of any one of claims 1 to 11, wherein the erythroid differentiation method occurs for a period of 14-18 days.

13. The potency assay of any one of claims 1 to 12, wherein the erythroid differentiation method occurs for a period of 14-17 days.

14. The potency assay of claim 1 or claim 2, wherein the fold change in Hemoglobin A expression is measured using ion-exchange HPLC.

15. The potency assay of claim 1 or claim 3, wherein the fold change in enucleated reticulocytes is measured using FACS.

16. A method for measuring relative potency of a drug product comprising:
a) transducing a sample of hematopoietic stem or progenitor cells from the subject having β-thalassemia and erythroid differentiating the transduced cells;
b) erythroid differentiating a sample of untransduced hematopoietic stem or progenitor cells from the subject having β-thalassemia;
c) quantifying fold change in Hemoglobin A (HbA) expression in the transduced erythroid cells compared to the HbA expression in the untransduced erythroid cells; and
d) quantifying fold change in the number of enucleated reticulocytes in the transduced erythroid cells compared to the number of enucleated reticulocytes in the untransduced cells,
wherein the transduced erythroid cells contain a lentiviral vector comprising a polynucleotide encoding a globin.

17. A method for measuring relative potency of a drug product comprising:
a) transducing a sample of hematopoietic stem or progenitor cells from the subject having β-thalassemia and erythroid differentiating the transduced cells;
b) erythroid differentiating a sample of untransduced hematopoietic stem or progenitor cells from the subject having β-thalassemia; and
c) quantifying fold change in Hemoglobin A (HbA) expression in the transduced erythroid cells compared to the HbA expression in the untransduced erythroid cells,
wherein the transduced erythroid cells contain a lentiviral vector comprising a polynucleotide encoding a globin.

18. A method for measuring relative potency of a drug product comprising:
a) transducing a sample of hematopoietic stem or progenitor cells from the subject having β-thalassemia and erythroid differentiating the transduced cells;
b) erythroid differentiating a sample of untransduced hematopoietic stem or progenitor cells from the subject having β-thalassemia; and
c) quantifying fold change in the number of enucleated reticulocytes in the transduced erythroid cells compared to the number of enucleated reticulocytes in the untransduced cells,
wherein the transduced erythroid cells contain a lentiviral vector comprising a polynucleotide encoding a globin.

19. The method of any one of claims 16 to 18, further comprising obtaining the hematopoietic stem or progenitor cells from the patient having β-thalassemia.

20. The method of any one of claims 16 to 19, wherein the hematopoietic stem or progenitor cells comprise CD34⁺ cells.

21. The method of any one of claims 16 to 20, wherein the hematopoietic stem or progenitor cells comprise CD133⁺ cells.

22. The method of any one of claims 16 to 21, wherein the hematopoietic stem or progenitor cells comprise CD34⁺CD38^{Lo}CD90⁺CD45RA-cells.

23. The method of any one of claims 16 to 22, wherein the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

24. The method of any one of claims 16 to 23, wherein the globin is a human β-globin, an anti-sickling globin, a human β^{A-T87Q}-globin, a human β^{A-G16D/E22A/T87Q_} globin, or a human β^{A-T87Q/K95E/K120E}-globin protein.

25. The method of any one of claims 16 to 24, wherein the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a GLOBE vector, a G-GLOBE vector, a βAS3-FB vector, or a derivative thereof.

26. The method of claim 16 or claim 17, wherein the fold change in Hemoglobin A expression is measured using ion-exchange HPLC.

27. The method of claim 16 or claim 18, wherein the fold change in enucleated reticulocytes is measured using FACS.

28. A potency assay for a gene therapy treatment for β-thalassemia comprising:
a) transducing a first sample of hematopoietic stem or progenitor cells from a subject having β-thalassemia with a lentiviral vector comprising a polynucleotide encoding a globin;
b) performing erythroid differentiation of the first sample of hematopoietic stem or progenitor cells;
c) performing erythroid differentiation of a second sample of untransduced hematopoietic stem or progenitor cells from the subject having β-thalassemia;
d) measuring fold change in Hemoglobin A expression in the transduced and the untransduced erythroid cell samples; and
e) measuring fold change in enucleated reticulocytes in the transduced and the untransduced erythroid cell samples,
wherein the potency of the gene therapy is assessed as the fold change in HbA expression and/or fold change in percent enucleated reticulocytes, in the first sample compared to the second sample.
